(19) United States
(12) Patent Application Publication
(10) Pub. No.: US 2014/0186306 A1
(43) Pub. Date: Jul. 3, 2014

(54) NOVEL AMPK AGONIST COMPOSITIONS AND METHODS OF USE

(71) Applicants: PAUL RONALD PLANTE, MIAMI BEACH, FL (US); SEBASTIAN MARIO MARCUCCIO, ENDEAVOR HILLS (AU); BARBARA JANE BADENOCH-JONES, BOWRAL (AU)

(72) Inventors: PAUL RONALD PLANTE, MIAMI BEACH, FL (US); SEBASTIAN MARIO MARCUCCIO, ENDEAVOR HILLS (AU); BARBARA JANE BADENOCH-JONES, BOWRAL (AU)

(21) Appl. No.: 14/036,442

(22) Filed: Sep. 25, 2013

Related U.S. Application Data
(60) Provisional application No. 61/707,822, filed on Sep. 28, 2012.
(60) Provisional application No. 61/786,144, filed on Mar. 14, 2013.

(51) Int. Cl. A61K 31/7056; A61K 45/06; A61K 31/728

(52) U.S. Cl. CPC A61K 31/7056; A61K 31/728; A61K 45/06
USPC 424/93.7; 514/43

(57) ABSTRACT

The invention relates to novel AMPK agonist containing compositions that are adapted for localised use in treating inflammation and/or pain due to disease or injury in a human or animal. The novel compositions of the invention are useful in the treatment of a variety of conditions including osteoarthritis (OA), synovitis, tendonitis, desmitis; cystitis, osteitis and laminitis. Routes of administration include intra-articular, direct injection into tissues, instillation, retrograde perfusion and topical. AMPK agonists which can be used include AICAR, metformin, phenformin, A-769662, resveratrol, berberine and polyphenols.

### RELATED APPLICATIONS

This provisional patent application claims the benefit of priority in US Provisional Application Ser. No. 61/703,913, entitled “NOVEL AMPK AGONIST CONTAINING COMPOSITIONS AND METHOD OF USE” filed on Sep. 21, 2012; and US Provisional Application Ser. No. 61/707,822, entitled “NOVEL AMPK AGONIST COMPOSITIONS AND METHODS OF USE” filed on Sep. 28, 2012.

### TECHNICAL FIELD

The present invention relates generally to novel AMPK agonist, (e.g., AICAR) containing compositions that are adapted for localized (i.e., non-systemic) use in treating inflammation and/or pain due to disease or injury, in a mammal, e.g., for localized delivery in treating musculoskeletal diseases in human and veterinary orthopedic procedures, namely for use in treating and/or preventing osteoarthritis (OA) and for normalization of the joint following a surgical procedure (e.g., arthroscopy) or for treating injuries such as tendonitis, ligament injury and the like. The novel compositions of the invention are also useful in the localized treatment of other inflammatory conditions that include but are not limited to those of certain other connective tissues such as the integumentary system, e.g., the epidermis (skin), transitional epithelium of the bladder (e.g., in treating cystitis such as interstitial or idiopathic cystitis) as well as localized treatment of inflammatory conditions of the central nervous system and peripheral nerves (e.g., Equine Protozoal Myeloencephalitis (EPM). Compositions of the invention are useful for localized treatment and/or prevention of pain as an anti-inflammatory and/or analgesic and/or local or regional anesthetic agent. In addition, the compositions can be used as a device for fluid replacement in (e.g., in a diarthrodial joint or the anterior chamber of the eye) or for use as a dermal filler (to treat wrinkles).

### BACKGROUND & SUMMARY OF THE INVENTION

References cited herein are hereby incorporated in their entirety by reference.

Inflammatory conditions and diseases (and associated pain) pose a considerable health burden in humans and in other animals. Whether created by a disease process or due to injury, such diseases and conditions are diverse, and for many of them, the treatment options are limited.

There are a number of inflammatory conditions that afflict mammals that can be treated by local drug administration. Local administration has a number of advantages over systemic administration. Local administration can often achieve higher concentrations in the specific target tissue/organ than can be achieved by systemic administration. The adverse side effects associated with systemic administration can often be avoided. Further, in human athletic competition and in the horse- and dog-racing industries, it is particularly beneficial to utilize local treatment of specific inflammatory conditions because AICAR, when given systemically, is a banned substance in many countries due to its performance-enhancing activity (Narkar et al., Cell, vol. 134, p 405, 2008; World Anti-Doping Agency. The 2012 Prohibited List. Available at: http://www.wada-ama.org/documents/world_anti-doping_program/wadp-prohibited-list/2012/wada_prohibited_list_{—}2012_en.pdf).

Local administration is cost effective because much smaller total doses are required than when a drug is administered systemically. This is particularly relevant to the treatment of large animals such as horses and cattle. Compared with orally administered drugs, local administration can also avoid drug losses associated with poor oral bioavailability, due to poor absorption, instability within the gastrointestinal tract or to high first pass effect and the like.

Prior to the present invention, however, there has been no teaching or suggestion that AICAR (or other AMPK agonists/activators) could be used either alone or in combination with at least one other agent wherein the AICAR or the combination is useful in the localized treatment and/or prevention of musculoskeletal inflammation and/or injury, e.g., the treatment and/or prevention of OA. Likewise, as set forth herein, prior to the present invention there has been no teaching or suggestion that AICAR (or other AMPK agonists) could be used for the localized treatment of other inflammatory conditions of a range of other tissues/organs. Similarly, prior to the present invention, there has been no teaching or suggestion that AICAR or other AMPK agonists could be used to provide local or regional analgesia or anesthesia.

AICAR (5-amino-1-β-D-ribofuranosyl-imidazole-4-carboxamide (CAS Registry Number: 2627-69-2)) is a naturally occurring intermediate in mammalian purine biosynthesis. AICAR and AICAR 5′-monphosphate (ZMP) are endogenous substances found in all mammals. AICAR is poorly absorbed after oral administration. Dixon et al. (J. Clin. Pharmacol., vol. 31, p 342, 1991) reported an oral bioavailability in humans of less than 5%. They also reported rapid plasma clearance in humans (2.2 L/h/kg).

AICAR enters cells through the adenosine transporter and is quickly phosphorylated to ZMP. Like AICAR, ZMP is a natural metabolic intermediate in the de novo purine biosynthetic pathway. Once in the cell, AICAR, following its conversion to ZMP, mimics the activity of AMP in activating AMP-activated protein kinase (AMPK) (Corton et al., Eur. J. Biochem., vol. 229, p 558, 1995). AICAR directly activates AMPK without altering adenine nucleotide ratios (see below).

AMPK is a highly conserved serine/threonine heterotrimeric protein consisting of a catalytic α subunit and regulatory 13 and y subunits (Oakhill et al., Acta Physiol., vol. 196, p 3, 2009). It is expressed ubiquitously and plays a key role in regulation of cellular energy homeostasis (reviews by Hardie et al., J. Physiol., vol. 574, p 7, 2006; Kola et al., Trends Endocrinol. Metab., vol. 17, p 205, 2006; Lage et al., Trends Mol. Med., vol. 14, p 539, 2008; Steinberg et al., Physiol. Rev., vol. 89, p 1025, 2009). AMPK is known as a master regulator by virtue of its central roles in many biological processes (see below).

Phosphorylation of the Thr¹⁷² residue within the α subunit of AMPK by upstream kinases is essential for activity of AMPK (reviewed by Carling, Biochimie, vol. 87, p 87, 2005). AMPK is activated allosterically by AMP which binds to the y subunit and by its upstream kinases promoting phosphorylation of Thr¹⁷² (Oakhill et al., Acta Physiol., vol. 196, p 3, 2009; Hardie, J. Cell Sci., vol. 117, p 5479, 2004; Bright et al., Acta Physiol., vol. 196, p 15, 2009).

AMPK is tightly regulated by the cellular AMP:ATP ratio and is activated by nutritional/metabolic stresses that increase this ratio. When activated, AMPK causes the switching on of ATP generating (catabolic) pathways, and the switching off of ATP consuming (anabolic) pathways that are not acutely necessary for survival. AMPK regulates many metabolic pathways in peripheral tissues by phosphorylating metabolic enzymes and affecting expression of genes involved in energy metabolism, including upregulation of the genes involved in mitochondrial biogenesis and oxidative phosphorylation (Bergeron et al., Am. J. Physiol. Endocrinol. Metab., vol. 281, p E1340, 2001, Ojuka, Proc. Nutr. Soc., vol. 63, p 275, 2004, Zong et al., Proc. Natl. Acad. Sci. USA, vol. 99, p 15983, 2002).

Whilst some of the effects of AICAR are due to its ability to stimulate AMPK, others appear not to be via this mechanism. There are a growing number of examples where the effects of AICAR are totally or partially independent of AMPK and it is becoming more and more evident that AICAR is a multi-target molecule resulting in complex effects (Daignan-Formier and Pinson. Metabolites, vol. 2, p 292, 2012).

The metabolic effects of AICAR have been the subject of many studies. Stimulation of AMPK increases glucose uptake in skeletal muscle, which is thought to be due to up-regulation of glucose transport via increased glucose transport protein (GLUT4) (Hayashi et al., Diabetes, vol. 47, p 1369, 1998; Kurth-Kraczek et al., Diabetes, vol. 48, 1667, 1999; Jessen et al., J. Appl. Physiol., vol. 94, p 1373, 2003). Activation of AMPK also increases lipid oxidation in skeletal muscle (Merrill et al., Am. J. Physiol. vol. 273, pE1107, 1997; Buhl et al., Diabetes, vol. 50, p 12, 2001), inhibits lipid synthesis (including fatty acid, cholesterol and isoprenoid synthesis) and hepatic gluconeogenesis, enhances lipid oxidation in the liver, and reduces lipogenesis and lipolysis in adipose tissue (reviews by Hardie, Gene Dev., vol. 25, p 1895, 2011; Salt et al., Diabetes, vol. 49, p 1649, 2000; Sullivan et al., FEBS Lett., vol. 353, p 1649, 1994). Together, these changes result in decreased plasma glucose and lipid levels and improved insulin sensitivity (Halseth et al., Biochem. Biophys. Res. Comm., vol. 294, p 798, 2002; Fiedler et al., Diabetologia, vol. 44, p 2180, 2001; Long et al., J. Clin. Invest., vol. 116, p 1776, 2006, Buhl et al., Diabetes, vol. 50, p 12, 2001; Merrill et al., Am. J. Physiol. vol. 273, pE1107, 1997; Cuthbertson et al., Diabetes, vol. 56, p 2078, 2007; Zhou et al., J. Clin. Invest., vol. 108, p 1167, 2001).

Chronic treatment of obese rodents with AICAR has been found to prevent the development of diabetes (Buhl et al., Diabetes, vol. 51, p 2199, 2002; Pold et al., Diabetes, vol. 54, p 928, 2005; Yu et al., Diabetologia, vol. 47, p 2012, 2004) and AICAR has also been found to enhance skeletal muscle endurance in sedentary mice (Narkar et al., Cell, vol. 134, p 405, 2008), partly by promoting mitochondrial biogenesis.

AICAR has anti-inflammatory activity (see below) and there could be a link between inflammation and insulin resistance. Increased secretion of pro-inflammatory cytokines from adipocytes and macrophages activates inflammatory pathways in insulin target cells, ultimately leading to decreased insulin sensitivity (Trayhurn and Beattie, Proc. Nutr. Soc., vol., 60, p 329; 2001; Rajala et al., Endocrinology, vol. 144, p 3765, 2003; reviewed by Hotamisligil, Nature, vol. 444, p 860, 2006). AICAR was found to decrease the production of various cytokines by human adipocytes (Lihn et al., Molec. and Cell Endocrinol., vol. 292, p 36, 2008; Sell et al., Biochem. Biophys. Res. Comm., vol. 343, p 700, 2006). This inhibition may play a role in AICAR-induced increase in insulin sensitivity. In addition to its effect on energy balance at the cellular level, AMPK also plays a key role in whole body energy homeostasis by integrating, at the hypothalamic level, nutrient and hormonal signals that regulate food intake and energy expenditure (Lage et al., Trends Mol. Med., vol. 14, p 539, 2008).

Although the role of AMPK in energy homeostasis is well known, the activity of AMPK during the inflammatory response has only been studied more recently. AICAR has anti-inflammatory effects in a wide range of tissues which appear to be mediated in part via its activation of AMPK. The acute effects of AICAR on inflammation have been revealed, for example, by measurement of expression of inflammatory response markers such as SAA cluster genes (acute phase response markers serum amyloid A (SAA1 and SAA2)) in the human hepatocarcinoma cell line HepG2 (Nerstedt et al., Diabetologia, vol. 53, p 2406, 2010).

A recent article has reviewed the molecular mechanisms underlying the anti-inflammatory effect of AMPK (Salt and Palmer, Expert Opin. Investig. Drugs, vol. 21, p 1155 2012). This review discussed inhibition of nuclear factor kappa-light-chain-enhancer of activated B cells (NF-κB) pathways, mitogen activated protein kinase (MAPK) pathways and of Janus kinase-signal transducer and activation of transcription (JAK-STAT) signalling.

NF-κB, a key pro-inflammatory transcription factor, is present in all nucleated cells and is thought to be critical for tumor necrosis factor (TNF)-α to mediate its pro-inflammatory effects. The NF-κB/Rel proteins regulate the expression of a large number of genes, many of which are important in inflammation. Examples include the genes for pro-inflammatory cytokines (e.g., interleukin (IL)-6, TNF-α and IL-β), chemokines (e.g. chemokine (C—X—C motif) ligand 2 (CXCL2)), adhesion molecules (e.g. vascular cell adhesion molecule-1 (VCAM-1)) and pro-survival/anti-apoptotic proteins (e.g. cellular FLICE-like inhibitory protein (cFLIP))(Siwkowski et al., Mol. Pharmacol. vol. 66, p 572, 2004; Micheau et al., Mol. Cell. Biol., vol. 36, p 5299, 2001).

Thus, AICAR inhibited activation of/signalling by NF-κB in a range of cell types, for example, bovine aortic endothelial cells, rat mesangial cells and HeLa cells (Katerelos et al., Immunol. Cell Biol., vol. 88, p 754, 2010), RAW 264.7 mouse macrophages (Kim et al., Clin. Exp. Allergy, vol. 37, p 1709, 2007), human vascular endothelial cells (HUVECs) (Hattori et al., Hypertension, vol. 47, p 1183, 2006; Cacicedo et al., Biochem. Biophys. Res. Comm., vol. 324, p 1204, 2004), primary cultures of rat astroglial cells (Ayasolla et al., J. Neuroinflamm., vol. 2, p 21, 2005) and cultured glial cells (Gini et al., J. Neurosci., vol. 24, p 479, 2004). Other transcription factors such as CCAAT/enhancer-binding protein alpha (C/EBP), cAMP response element-binding protein (CREB) and activator protein 1 (AP-1) are also inhibited by AICAR (Ayasolla et al., J. Neuroinflamm., vol. 2, p 21 2005; Gini et al., J. Neurosci., vol. 24, p 479, 2004).

Cytokines, particularly TNF-α, play major roles in various inflammatory diseases, and AICAR has been shown in vitro to inhibit their production in a wide range of stimulated cells/tissues, for example, cultured rat microglial cells (Labuzek et al., Neurotoxicol., vol. 31, p 134, 2010), a human retinal pigment epithelial (RPE) cell line (Qin et al., Investig. Ophthalmol. Vis. Sci., vol. 49, p 274, 2008), and RAW 264.7 murine macrophages (Jhun et al., Biochem. Biophys. Res. Comm., vol. 318, p 372, 2004). AICAR also blocked lipopolysaccharide (LPS)- and Aβ peptide-induced expression of the cyclooxygenase-2 (COX-2) gene in primary rat astroglial cell cultures (Ayasolla et al., J. Neuroinflamm., vol. 2, p 21, 2005). There was evidence that these effects in RPEs and RAW 264.7 macrophages were independent of AMPK activation.

AICAR has been found to affect the production by various cell types of nitric oxide (NO), a key signalling molecule, particularly in the cardiovascular and nervous systems. Thus, AICAR suppressed NO release in human chrondrocytes and mouse cartilage explants stimulated with IL-1β and TNF-α (Terkeltaub et al., Arthritis & Rheumat., vol. 63, p 1928, 2011) and blocked expression of the inducible nitric oxide synthase (iNOS) gene induced in primary rat astroglial cell cultures by LPS and Aβ peptide (Ayasolla et al., J. Neuroinflamm., vol. 2, p 21, 2005). However, it did not affect NO production in LPS-stimulated cultured microglial cells (Labuzek et al., Neurotoxicol., vol. 31, p 134, 2010).

Cell adhesion molecules play a critical role in inflammation, and AICAR has been found in vitro to inhibit these molecules in endothelial/epithelial cells. Thus, AICAR inhibited TNF-α and IL-1β-induced increases in gene expression of VCAM-1, endothelial-leukocyte adhesion molecule 1 (E-selectin) and intercellular adhesion molecule-1 (ICAM-1) in cultured HUVECs (Hattori et al., Hypertension, vol. 47, p1183, 2006) and of ICAM-1 in RPEs (Qin et al., Investig. Ophthalmol. Vis. Sci., vol. 49, p 274, 2008) and palmitate-induced increases in VCAM-1 expression in cultured HUVECs (Cacicedo et al., Biochem. Biophys. Res. Comm., vol. 324, p 1204, 2004). In the RPEs, the effect appeared to be independent of AMPK activation.

In some diseases, reactive oxygen species (ROS) can increase disease severity by promoting the activities of nuclear factors such as NF-κB. AMPK activated by AICAR is involved in regulation of ROS (Labuzek et al., Neurotoxicol., vol. 31, p 134, 2010). It has been reported that activation of AMPK by AICAR reduced both translocation to the cell membrane and phosphorylation of a cytosolic component of NADPH oxidase (Alba et al., FEBS Lett., vol. 573, p 219, 2004). Reducing the production of ROS in the cell has been suggested as a mechanism by which AICAR suppresses apoptosis (Kim et al., J. Pharmacol. Sci., vol. 106, p 394, 2008). Ayasolla et al. (J. Neuroinflamm., vol. 2, p 21, 2005) found that AICAR blocked expression of the superoxide dismutase, MnSOD gene, and inhibited ROS generation and depletion of glutathione in primary rat astroglial cell cultures induced by LPS and Aβ peptide (the latter is known to alter cellular redox thereby triggering downstream kinase cascades leading to inflammation).

AICAR is also involved in the regulation of cell proliferation and apoptosis. Several reports show that AICAR can exhibit either anti-apoptotic or pro-apoptotic effects in different cell types, via both AMPK-dependent and independent mechanisms (Stefanelli et al., Biochem. Biophys. Res. Comm., vol. 243, p 821, 1998; Campas et al., Blood. v. 1. 101, p 3674 2003; Lopez et al., Biochem. J., vol. 370, p 1027, 2003). AICAR has antiproliferative effects which have been demonstrated in several tumour cell lines, for example, PC-3, MCF-7, C6 glioma, U87MG, K-562 and CEM (Meli et al., J. Med. Chem., vol. 49, p 7721, 2006). It has been shown to specifically induce apoptosis of aneuploid cells (Tang et al., Cell, vol. 144, p 499, 2011). It has been used for the treatment of acute lymphoblastic leukemia (Cronstein and Kamen, J. Pediat. Haematol./Oncol., vol. 29, p 805, 2007) and B-cell chronic lymphocytic leukemia (Santidrian et al., Blood, vol. 116, p3023, 2010).

AMPK plays an important role in proliferation and differentiation of T-lymphocytes (See review by Salt and Palmer, Expert Opin. Investig. Drugs, vol. 21, p 1155 2012). AICAR inhibited the in vitro differentiation of T helper (Th) 1 and Th17 cells (Bai et al., J. Pharmacol. Exp. Ther., vol. 333, p 717, 2010; Nath et al., J. Immunol., vol. 182, p 8005, 2009). Blockade of Th17 development by AMPK is particularly important as deregulated expansion of Th17 cells is a key driver of autoimmune conditions such as colitis and rheumatoid arthritis (Bai et al., Biochem. Pharmacol., vol. 80, p 1708, 2010).

AICAR is known to protect tissues from damage induced by ischemia, trauma and sepsis (Zhao et al., Cardiovasc. Res., vol. 29, p 495, 1995, Fabian et al., Surgery, vol. 119, p 302, 1996, Davis et al., J. Trauma, vol. 49, p 892, 2000). One mechanism by which AICAR might protect tissues under ischemic conditions is to increase the availability of adenosine in tissues (Gruber et al., Circulation, vol. 80, p 1400, 1989). Additionally, part of this protective effect was reported to be correlated with its anti-inflammatory effects (Mullane and Bullough, J. Mol. Cell. Cardio., vol. 27, p 1041, 1995, Melton et al., Crit. Care Med., vol. 27, p 565, 1999). AICAR's anti-apoptotic effects might also play a role in this protection. AICAR has been investigated in vivo in reperfusion injury of the heart (Manango et al., J. Am. Coll. Cardiol., vol. 48, p 206, 2006) and has undergone Phase III clinical trials for the prevention of adverse cardiovascular outcomes in patients undergoing coronary artery bypass graft surgery (Leung et al., Anesth. Anal., vol. 78, p 420, 1994).

In IL-1β and TNF-α-stimulated mouse cartilage explants, AICAR inhibited glucosaminoglycan release and in human chrondrocytes in vitro, AICAR inhibited IL-1β- and TNF-α-induced suppression of proteoglycan synthesis (Terkeltaub et al., Arthritis & Rheumat., vol. 63, p 1928, 2011). In both the cartilage explants and cultured chondrocytes, release of matrix metalloproteinases (MMPs), MMP-3 and MMP-13, was inhibited by AICAR.

AMPK signalling might also play a role in skeletal physiology (Shah et al., Bone, vol. 47, p 309, 2010). Thus, AICAR inhibited proliferation and alkaline phosphatase activity in the rat osteosarcoma cell line ROS 17.28, and increased trabecular bone nodule formation in primary cultures of osteoblasts from rat calvaria.

In mouse sensory neurons cultured in the presence of nerve growth factor, Melemedijian et al. (Molec. Pain, vol. 7, p 70, 2011) reported that treatment with AICAR suppressed the activity of two pathways that are believed to play an important role in neuropathic pain, the mammalian target of rapamycin complex (mTOR) (Geranton et al., J. Neurosci., vol. 29, p 15017, 2009) and the extracellular signal-related kinases (ERK) pathways (Song et al., Acta Pharmacol. Sin., vol. 26, p 789, 2005).

Except for studies of metabolic effects, there have been relatively few studies investigating the effects of AICAR in experimental animals in vivo. The effects of AICAR in reducing myocardial ischemic injury in several animal models have been noted (Mullane, Cardiovasc. Res., vol. 27, p 43, 1993). AICAR has been shown to be therapeutically effective in experimental autoimmune encephalitis (Nath et Immunol, vol. 175, p 556, 2005). AICAR inhibited airway inflammation and airway hyperresponsiveness in vivo in 2 murine models of asthma (ovalbumin-induced and ovalubumin plus polyinosinic-polycytidylic acid-induced, but particularly the latter) through inhibition of the influx of inflammatory cells and cytokines into the lung (Kim et al., Clin. Exp. Allergy, vol. 37, p 1709, 2007). Bai et al. (Biochem. Pharmacol., vol. 80, p 1708, 2010) showed that AICAR was therapeutically effective in a murine model of inflammatory bowel disease (2,4,6-trinitrobenzene sulfonic acid (TNVS)-induced colitis).

As set forth herein, the current compositions comprised of AICAR (or a suitable AMPK agonist) are useful in the localized treatment of inflammatory conditions/diseases where such treatment is practicable. In particular, the present invention contemplates novel compositions comprised of AICAR or another suitable AMPK agonist (activator) adapted for localized delivery that can be used to treat and/or prevent inflammation or pain associated with musculosleletal disease or injury. For example, inflammatory conditions of a joint that can be treated by direct intra-articular injection of the instant AICAR containing compositions, include but are not limited to OA/degenerative joint disease (DJD), synovitis, capsulitis, and bursitis, and the like.

OA is a degenerative and chronic inflammatory disease characterized by loss of hyaline articular cartilage, and associated with varying degrees of synovitis, subchondral bone change and osteophyte formation. OA can originate from various causes but generally an inflammatory process begins in the synovium, cartilage, joint capsule or subchondral bone and quickly initiates a cascade of inflammatory mediators (See review by Goodrich and Nixon, The Vet. J., vol. 171, p 51, 2006). Hyaluronic acid (HA) in the synovial fluid is degraded due to enzymes and free radicles released by synoviocytes. This results in a reduction in the viscosity of the synovial fluid and therefore a reduction in its lubricating capability. This reduced viscosity, as well as the release of degradative enzymes (most notably the metalloproteinases, e.g. collagenase, stromelysin and gelatinase) into the synovial fluid, results in articular cartilage breakdown. The inflammatory process involves the release of arachidonic acid metabolites by the synoviocytes, in particular the prostaglandins of the E (PGE₂) and I (PGI₂) series which initiate pain.

Localized inflammatory conditions of other connective tissues that can be treated by injection of the AICAR containing compositions include for example, other musculoskeletal injuries such as injury to tendons and ligaments, e.g. tendonitis, tendinosis, tenosynovitis, tendon strains or tears (rupture or partial rupture), suspensory ligament desmitis, strains and/or tears. Suspensory ligament injuries are particularly common in horses. Such injuries initiate a local inflammatory response that can be treated by local injection of the AICAR containing compositions. Other connective tissue inflammatory conditions that can be treated by local injection of the novel compositions also include fasciitis. Inflammatory conditions of bone that can be treated by injection into adjacent soft tissue of the AICAR containing compositions include but are not limited to exostoses, osteitis, periostitis, and the like. AICAR may be useful as an antiinflammatory agent to treat septic arthritis, given its lack of immunosuppressant activity. It may also be beneficial to infiltrate the periosteum at the site of bone surgery and to use in the treatment of navicular disease.

Likewise, certain CNS diseases and injuries can be treated via localized delivery of the compositions provided by the invention. EPM, for example, is a CNS disease of horses that is caused by the parasite Sarcocystis neurona. The horse is an aberrant or dead end host for this apicoplexan parasite which has an affinity for the brain and spinal cord (particularly the grey matter) of affected equids. Several agents such as diclazuril have demonstrated at least some measure of efficacy in treating affected animals for removal of the organism, however, relapses following treatment are common and results are variable (See, e.g., U.S. Pat. No. 5,883,095 to Tobin et al). Likewise, certain systemic treatments aimed at reducing the inflammation produced by these organisms such as systemic administration of corticosteroids and NSAIDs have been tried as a means to reduce the inflammation of the affected CNS tissues, often with poor to limited efficacy and often with harmful side effects due to systemic administration of these agents.

Therefore, there exists a need in the art for an agent that can be utilized via localized delivery to reduce the inflammatory effects of the parasite without deleterious side systemic effects. Prior to the present invention, there has been no teaching or suggestion that the inflammation caused by such diseases could be treated via localized delivery, (e.g., by epidural injection into the cerebral spinal fluid (CSF)) utilizing the compositions provided by the instant invention.

Laminitis is an inflammatory condition of the feet of horses and other equine species that is characterized by inflammation of and loss of blood flow to the sensitive lamina of the hoof wall (See, e.g., “Making Sense of Laminitis”, Anderson, www.thehorse.com, Jan. 16, 2013). Loss of blood flow to the sensitive lamina of the hoof wall results in cell death and separation of the sensitive (oxygenated) lamina from the insensitive (keratinized) lamina of the hoof wall. Precipitating factors of laminitis are many and varied including: infection; stress; metabolic disorders such as diabetes; iatragenic factors, such as glucocorticoid overdose; endotoxins and the like. However, the end result is the same, inflammation and death of the sensitive lamina of the hoof wall resulting in severe pain and rotation and/or sinking of the hoof wall as the lamina separate. The inflammation and pain associated with laminitis is often treated systemically with NSAIDs and the like which are associated with side effects as mentioned above. Therefore, there still exists a need in the art for a treatment of the inflammation and pain associated with laminitis that can be administered locally to reduce inflammation and provide relief from pain, e.g., analgesia, without concomitant unwanted systemic side effects. Infections of the hoof, distal phalanges, coronary band, navicular bursa and related structures have been treated with retrograde perfusion (regional intravenous perfusion) of the affected tissues. (See, e.g., Murphey et al., J. vet Pharinacol. Therap. 22, 68-71 1999). This technique of retrograde perfusion could be used to administer the compositions provided by the instant invention to treat the inflammation and pain associated with laminitis.

Inflammatory conditions of the eyes/eyelids that can be treated by local application (topical or injection) of the AICAR containing compositions include but are not limited to uveitis, scleritis and iritis, retinitis, choroiditis, chorioretinitis, blepharitis and non-infective conjunctivitis. Inflammatory conditions of skin that can be treated by topical application of the AICAR containing compositions include but are not limited to dermatitis (eczema), psoriasis, rosacea and acne. Mange (demodectic or sarcoptic) is an inflammatory disease in dogs that produces intense pain, puritis and chronic inflammation, and the compositions set forth herein are useful in relieving the inflammation, pain and/or puritis associated with such conditions.

Inflammatory conditions of the bladder that can be treated by direct instillation (e.g., intravesicular administration) of the AICAR containing compositions include but are not limited to interstitial cystitis, iatrogenic cystitis (e.g., as a sequela from treatment with chemotherapeutic agents or radiation treatment of bladder or prostate cancer), hemorrhagic cystitis (e.g., in recipients of allogeneic hematopoietic stem cell transplants) in humans, and idiopathic cystitis in cats.

Other localized situations involving an inflammatory process where localized treatment with the AICAR containing compositions would be indicated include wound healing, either accidental dermal lesions or post operative lesions to any part of the body. Likewise, numerous injury/inflammatory conditions of muscle that can be treated by localized injection of the AICAR containing compositions include but are not limited to myositis, pulled and/or strained muscles, and the like. Inflammatory conditions of peripheral nerves that can be treated by local injection of the AICAR containing compositions include but are not limited to neuritis, intervertebral disc disease, EPM and the like. Inflammatory airway disease can be treated by inhalational administration of the AICAR containing compositions, including but not limited to exercise-induced pulmonary hemorrhage (EIPH) and heaves in horses, chronic obstructive pulmonary disease (COPD), and the like.

There are a number of diseases, generally believed to be autoimmune diseases, which result in inflammation of connective tissue/joints/skin, including but not limited to rheumatoid arthritis, systemic lupus erythematosis, relapsing polycondritis, scleroderma and mixed connective tissue disease. While these diseases generally result in systemic inflammatory changes, there may be instances where localized treatment of the disease/disease symptoms with the AICAR containing compositions is warranted.

### DETAILED DESCRIPTION OF THE INVENTION

Prior to the present invention there existed a need in the art for a composition comprised of AICAR or a suitable AMPK agonist/activator that could be used locally within the body for the treatment of injuries and the treatment and/or prevention of a variety of inflammatory conditions in e.g., in human and animal athletes thereby avoiding the effects of systemic AICAR administration. The limited oral bioavailability of AICAR and the performance enhancing effects seen with systemic administration in human and animal athletes, make systemic treatment with AICAR both cost prohibitive and an undesirable treatment for many conditions that affect athletes.

By localized delivery, localized administration or localized treatment is meant direct administration into or adjacent to the target tissue in need of treatment. Localized delivery can, e.g., be by direct injection into the target tissue such as intra-articular injection for treatment of disease and/or injury to a diathrodial joint or by injection into the tissue adjacent to the target joint, e.g., peri-articular injection. Likewise, localized delivery can include direct injection into the margins of a surgical incision or open wound or by injection into tissue adjacent the margins of a surgical incision or open wound. Pain and inflammation of joints can therefore be treated by direct intra-articular injection or peri-articular injection utilizing the compositions provided by the invention, while pain associated with a surgical incision or open wound can be treated by direct injection of the compositions provided by the invention into or adjacent to the margins of the incision/wound. Likewise, disease and/or injury to other musculoskeletal tissue such as tendons, ligaments or bone can be treated by direct injection into the tendon or ligament at the site of injury. Alternatively, injecting adjacent to an injured tissue is also contemplated, such as infiltration of the compositions of the invention around an inflamed nerve root or an osseous injury, e.g., bucked shins (periostitis) or osteophyte or bone spur formation.

In addition, localized delivery or treatment is meant to include but not be limited to topical administration, transmucosal delivery, inhalation therapy, epidural injection, intravesicular administration, intrauterine administration and the like. Given the teachings provided by the present invention, one of skill in the art can appreciate that treatment of a variety of tissues within the mammalian body are possible utilizing the compositions of the invention with methods of localized delivery, localized treatment or localized administration that are known in the art depending upon the target tissue that is in need of treatment.

The term musculoskeletal is meant to include but not be limited to bone, cartilage, tendons, ligaments, connective tissue and the like. Connective tissue is meant to include but not be limited to musculoskeletal tissue and the dermis and epidermis as well as loose connective tissues, e.g., areolar connective tissue.

In particular, the present invention provides compositions adapted for intra-articular administration that will inhibit inflammatory processes within an OA affected joint. The novel compositions of the invention can be used in methods provided by the instant invention to treat or prevent the onset or progression of OA in a preselected joint in a mammal. The joint can be any of the diarthrodial joints in a mammal including but not limited to, e.g., carpal, metacarpal, femoro-tibial (knee), tarsal, metatarsal, scapula-humeral (shoulder), humero-radial (elbow) and the like. The tempormandibular joint (TMJ) is a specific joint that in humans is prone to disorders, in particular, TMJ OA, that would be suitable for treatment with proposed the novel compositions. The mammal can be any mammal including, but not limited to man, horses, dogs, cats, sheep, goats, cows, camelids and non-human primates.

Prior to the present invention there existed a need in the art for a novel composition that can function to provide a protective mechanism for cartilage to aid an OA-affected or post-surgical joint in returning to normalization. In particular, the present invention provides compositions adapted for intra-articular administration that will reduce the inflammatory process within an OA affected joint and thereby protect cartilage from further degradation, reduce pain, reduce subchondral bone change and osteophyte formation and also prevent the onset or progression of OA in a preselected joint in a mammal.

In a presently preferred embodiment, the invention provides a composition adapted for localized delivery (e.g., intra-articular administration) for the treatment of disease or injury in a mammal comprised of a therapeutically effective amount of AICAR or a suitable AMPK agonist. In one embodiment the therapeutically effective amount of AICAR is from between about 0.01 mg/ml to about 400 mg/ml AICAR. In another embodiment, the therapeutically effective amount is from between about 1 mg/ml and about 20 mg/ml.

There is a need in the art for a composition that can be used to simultaneously act as an anti-inflammatory agent and a local analgesic or anesthetic to treat both inflammation and pain associated with a variety of conditions such as synovitis and the like. By “analgesic” is meant an agent that lessens or eliminates sensibility to pain. By “local anesthetic” is meant an agent that causes the loss of a conscious perception of pain, by blocking or inhibiting transmission of impulses from the peripheral nervous system to the central nervous system via nociceptor pathways. Local anesthetics are therefore analgesics that eliminate pain or reduce pain by blocking transmission of the pain impulses to the brain.

In addition, there is a need in the art for a composition that can be utilized to simultaneously act as an analgesic or local anesthetic and an anti-inflammatory at a surgical incision site or open wound. In fact, the International Veterinary Academy of Pain Management is in the process of adopting a position statement recommending that every surgical procedure, including those that require general anesthesia, utilize a local anesthetic to induce analgesia at the surgical site for prevention of pain and overall improvement of surgical outcome (Mark E. Epstein, DVM, DABVP (C/F), DAAPM, CVPP International Veterinary Academy of Pain Management presentation at North American Veterinary Conference (NAVC), FL, 2013). The blockade of pain impulses originating in the periphery and preventing them from reaching the central nervous system has several positive consequences including, smoother induction and recovery, a decrease in the minimum alveolar concentration (MAC) of all anesthetic gases during anesthesia and possible anti-inflammatory effects post surgically (See, Epstein, Proceedings NAVC, p. 2167 (2013)).

We have surprisingly found that AMPK agonists such as AICAR at certain doses can be used via localized delivery to treat inflammatory conditions and act as an analgesic (to treat pain) at the same time. This dual activity (analgesic and anti-inflammatory), at certain doses, can be used to advantage in a variety of different situations where treatment of both inflammation and pain is required or is beneficial, e.g., at a surgical incision to prevent pain and treat inflammation or at the margins of an open wound. Therefore methods of simultaneous treatment of inflammation and/or for providing local analgesia are contemplated. For instance, in the examples set forth herein below, treatment of both inflammation and inducement of analgesia were achieved in certain cases by direct injection (localized delivery) of a composition comprised of about 1.3 mg/ml of AICAR. It can be appreciated by one of skill in the art given the teachings provided herein that the compositions of the invention may be used as an anti-inflammatory agent or as an analgesic depending upon the need and condition of the mammal being treated.

AICAR(CAS Registry Number: 2627-69-2) is presently known and is also referred to as AICA riboside, 5-Amino-1-β-D-ribofuranosyl-1H-imidazole-4-carboxamide, 5-Amino-1-β-D-ribofuranosylimidazole-4-carboxamide; Ribofuranosyl-5-amino-4-imidazolecarboxamide; 1-Ribosyl-4-carboxamido-5-aminoimidazole; 5-Amino-1-ribosyl-4-imidazolecarboxamide; 5-Amino-4-imidazolecarboxamide ribofuranoside; 5-Amino-4-imidazolecarboxamide riboside; 5-Aminoimidazole-4-carboxamide 1-(β-D-ribofuranoside); AIC-Riboside; Acadesine; Arasine; GP 1-110; NSC 105823, ARA 100, Acarda, and the like.

The chemical structure of AICAR is shown below:

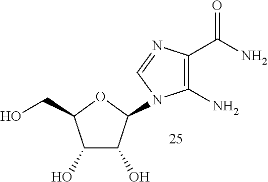 [SMILES: NC(=O)c1ncn([C@@H]2O[C@H](CO)[C@@H](O)[C@H]2O)c1N]

While AICAR is the presently preferred AMPK agonist that is useful in the compositions of the invention, it is contemplated that other AMPK agonists (activators) are suitable for use, including but not limited to, metformin, phenformin, A-769662, resveratrol, thiazolidindiones (including rosiglitazone, pioglitazone and troglitazone), D942 527847, nootkatone, berberine, dhberberine, polyphenols (including S17834, piceatannol, CA-4, EGCG, TF1, TF2, TF3) WS070117, leptin, adiponectin, DRL-16536, BG800, MT-39 series of structures, salicylic acid (its salts and prodrugs), triterpenoids (including cucurbitane triterpenoids and ginsenoside Rg3) and the like.

Prodrugs, salts and metabolites or other derivatives of AICAR and other AMPK agonists are known to one of skill in the art and are contemplated by the invention for use in the compositions set forth herein for treating and/or preventing OA in man or animals or for use in the other compositions or methods set forth herein. Examples of known AICAR prodrugs include but are not limited to those described in U.S. Pat. No. 5,132,291 (1992, H. E. Gruber, Gensia Pharmaceuticals Inc.) and U.S. Pat. No. 5,658,889 (1997, H. E. Gruber, Gensia Pharmaceuticals Inc.) hereby incorporated by reference herein.

One embodiment of the invention provides a composition adapted for intra-articular administration useful for the treatment and/or prevention of, e.g., OA, synovitis, caspsulitis, bursitis and the like in a mammal comprised of a therapeutically effective amount of a suitable AMPK agonist (activator) alone or in combination with a suitable HA. The suitable AMPK agonist can be any of the known AMPK agonists including but not limited to AICAR, metformin, phenformin, A-769662, resveratrol, thiazolidindiones (including rosiglitazone, pioglitazone and troglitazone), D942, S27847, nootkatone, berberine, dhberberine, polyphenols (including S17834, piceatannol, CA-4, EGCG, TF1, TF2, TF3), WS070117, leptin, adiponectin, DRL-16536, BG800, MT-39 series of structures, salicylic acid (its salts and prodrugs), triterpenoids (including cucurbitane triterpenoids and ginsenoside Rg3) and the like. However, the presently preferred AMPK agonist is AICAR.

As set forth above, it is contemplated that the compositions provided by the invention can be comprised of a suitable AMPK agonist alone or in combination with another suitable agent. Examples of other suitable agents that may be combined with the AMPK agonist (e.g., AICAR) include but are not limited to: HA; sulfated polysaccharides (e.g. chondroitin sulfate, polysulfated glycosaminoglycans (PSGAGs) (e.g. ADEQUAN®), pentosan polysulfate (e.g. CARTOPHEN, the semi-synthetic polysaccharide, IPS and glycosaminoglycan peptide complex, (e.g. Rumalon)); N-acetyl-D-glucosamine; corticosteroids (e.g., methylprednisolone acetate, betamethasone, triamcinalone acetonide, isoflupredone acetate, dexamethasone and the like); and non-steroidal anti-inflammatory agents (NSAIDs) (e.g. bufexamac, ketoprofen, naproxen, ibuprofen, meloxicam, flunixin meglumine, carprofen, phenylbutazone, ketoprofen, firocoxib, deracoxib and the like). Other suitable agents can include: local anesthetics (e.g., mepivacaine, lidocaine and the like); superoxide dismutase; dimethyl sulfoxide (DMSO); autologous conditioned serum (ACS) or autologous conditioned plasma; platelet rich plasma (PRP); IL-1 receptor antagonist protein (TRAP) I, IRAP II and the like; stem cells, including but not limited to mesenchymal stem cells (MSC), bone marrow derived stem cells, umbilical derived stem cells, stem cells from cultured stem cell lines and the like; chondrocytes; insulin like growth factor-1 (IGF-1); lubricin/proteoglycan 4/PRG4; gene therapy products; nanoparticles, and combinations thereof.

Accordingly, the invention provides methods of treating and/or preventing disease or injury to tissues of the musculoskeletal system in a mammal, e.g., tendons, ligaments or joints (for example, OA in a mammal) comprised of administering to the mammal a therapeutically effective amount of a composition comprised of an AMPK agonist such as AICAR. In one embodiment of the invention, the composition optionally includes a suitable HA. One of skill in the art can appreciate that, depending upon the condition being treated and the severity of the disease condition or injury, various treatment regimens can be applied in the methods of treatment contemplated by the invention. For example, intra-articular injection of the compositions of the invention can be repeated as needed, e.g., every two weeks for a three injection series similar to treatment protocols with HA.

The invention provides compositions useful for the treatment and/or prevention of damage to connective tissues (including, but not limited to ligaments and tendons and diarthrodial (synovial) joints) and, in particular, treatment of traumatic synovitis, inflammation of the synovial membrane, and damage to the articular cartilage of the joint, namely OA. Specifically provided are compositions formulated for intra-articular and/or localized parenteral (e.g., peri-articular) use in the treatment and/or prevention of traumatic synovitis and/or damage to articular cartilage or connective tissues. Compositions adapted specifically for post surgical joint lavage or treatment and/or prevention of inflammatory arthritis, OA/DJD are also provided.

The invention also provides methods for treatment of neurologic disease or injury such as inflamed nerves, degenerative disc disease and the like. In particular, the invention also provides for the treatment of EPM in horses by localized delivery of the compositions of the invention, providing a huge advantage over conventional treatment in that the compositions of the invention are not known to be immunosuppressive at therapeutic dosages. In one embodiment, the invention provides a composition for the treatment of EPM that is adapted for epidural administration and can be administered directly into the cerebrospinal fluid (CSF) and a corresponding method of treatment for EPM and associated CNS inflammation comprising localized delivery of a therapeutic amount of the compositions of the invention directly into the CSF (e.g., AICAR or a suitable AMPK agonist).

In another embodiment, the invention provides a method of treatment of the inflammation and pain associated with laminitis via retrograde perfusion comprising administering a therapeutic amount of a composition comprised of AICAR or a suitable AMPK agonist via distal regional perfusion to provide localized delivery to the hoof wall and affected tissues.

It would be appreciated by one skilled in the art that adverse side effects which may be associated with systemic administration of AICAR can be avoided by local administration. In addition, given the much smaller doses used/required for the present treatments, problems associated with the perceived performance-enhancing activities of AICAR in human athletic competition and in the horse-racing and dog-racing industries can be largely avoided. For comparison, Narkar et al. (Cell, Vol. 134, p 405, 2008) found a performance-enhancing activity of AICAR in mice at an oral dose of 500 mg/Kg/day over a 4 week period. In examples of the present invention set forth below, amounts as low as 3.25 mg of AICAR were used per joint in horses of 450 Kg or above (less than 0.0072 mg/Kg). In many cases only one treatment is required.

It can be appreciated by one of skill in the art that the compositions provided by the present invention can be used for a variety of other applications including, e.g., lubrication of an instrument such as a catheter or endoscope prior to performing a surgical or other procedure (e.g., lubricating the lumen and/or exterior surface of a catheter or endoscopic instrument prior to insertion or placement of the instrument at the treatment or surgical site).

Likewise, the present invention contemplates methods of treating respiratory conditions in a mammal by localized delivery of the compositions of the invention to the respiratory tract, e.g., by inhalation or nebulization therapy. In addition, the compositions of the invention can be formulated into lotions, creams, ointments and the like for localized (topical) administration to treat inflammatory conditions of the skin. Preparations for use as eye drops, ear drops and solutions for intrauterine infusion are also contemplated.

Given the teachings provided by the invention, other uses for the compositions of the invention will be apparent to one of skill in the art and are contemplated by the invention.

### EXAMPLES

The present invention provides novel compositions of matter comprised of AICAR that are adapted for localized delivery and treatment of musculoskeletal diseases and injuries. In the examples below the novel or test composition indicated as NOVEL was comprised of 1.3 mg/ml solutions of AICAR. The purpose of this study was to evaluate the clinical response of racing and non-racing horses with joint and/or soft tissue lameness to various doses of AICAR, either injected intra-articularly or locally infiltrated into the affected soft tissue structure. Clinical cases actively in training for their sport, or racing, demonstrating lameness were selected by various attending veterinarians. Selection criteria for the clinical cases entered into the study were: male or female, any age, with lameness demonstrating heat, effusion, and pain upon flexion and/or palpation. In most cases, multiple joints or soft tissue structures per animal were treated. In some cases, joints and soft tissue structures were injected simultaneously.

Addition of 0.5 ml of an aminoglycoside antibiotic per 2.5 ml vial of AICAR was left up to the discretion of the attending veterinarian. Prior to the treatment, each case was graded for lameness, swelling and heat, and pain. Each injected joint received either one, 1.3 mg/ml, 2.5 ml dose (3.25 mg) for a small joint, or two, 2.5 ml dose vials for large (stifle) joints. Soft tissue structures, including tendons and tendon sheaths, suspensory ligaments, exostoses (splints), periosteal osteitis, and perineural injections were done with multiple vials of the AICAR. The dose was dependent upon the perceived surface area to be treated. The horses were re-examined at various times post-injection as most horses were stabled at locations remote to the veterinarians. All cases were followed up by telephone calls to trainers/caretakers within 24 hours. No adverse events were reported with the exception of transient swelling of a fetlock that was traumatically injected. This resolved without treatment. No part of the study was conducted blind. The study began in mid-June, 2012 and is ongoing. No other medications were administered to the horses included in this study.

### E. Case Examples

I. Signalment (Sx): Horse No: 1 (Massachusetts, U.S.), 6 year old Standardbred (SB) pacing mare.

History (Hx): Poor racing performance.

Clinical Signs (CS): Bilateral effusion at anterior aspect of front coffin joints. 1/4 pain to hoof testers; 1/4 lameness, swelling, pain.

Diagnosis (Dx): Synovitis of front coffin joints.

Treatment (Tx): 2.5 cc NOVEL and 0.5 cc gentamicin/joint (8/22/12).

Results (Rx): 7 days post-injection: negative pain, swelling, lameness. Raced and won in 1:56.2. (Previously raced approx. 1:59).

II. Sx: Horse No: 2 (Rome, Italy), 6 year old SB trotting mare.

Hx: Chronic generalized lameness; poor performance.

CS: 4/5 lame RF localized to fetlock and coffin joints.

Dx: Radiographs reveal osteoarthritis of RF fetlock. Synovitis of front fetlocks, coffins, and distal hocks.

Tx: 2.5 cc NOVEL and 0.5 cc amicacin×6 joints: LF and RF fetlocks, LF and RF coffin joints, LTMT and RTMT joints (7/30/12).

Rx: 48 hours post-injection: 50% improvement in pain and lameness.
96 hours post-injection: 80% improved. Raced and finished 4^{th}, 1:58.
3 weeks post-injection (8/21/12): no swelling, pain, or lameness. Raced and won, 1:59.
   III. Sx: Horse No: 3 (Rome, Italy), 6 year old SB trotting gelding.

Hx: Prior bilateral front lateral splint bone surgery resulting in bilateral, lateral suspensory branch desmitis. Chronic synovitis bilateral front fetlocks.

CS: 4.5/5.0 lame and painful bilaterally in front.

Dx: Bilateral lateral branch suspensory desmitis and front fetlock synovitis.

Tx: 2.5 cc NOVEL per each front fetlock.
3×2.5 cc AICAR per each front lateral suspensory branch (7/11/12).

Rx: 24 hours post-injection: 50% improved per trainer.
48 hours post-injection: Raced. Second.
5 days post-injection: Per veterinarian, sound and suspensories smaller.
8 days post-injection: Re-exam: LF fetlock non-painful
LF lateral suspensory non-painful, near normal size.
RF fetlock flexes +1 painful
RF lateral suspensory 50% smaller and 50% less pain.
9 days post-injection: Raced and won! Sound post-race.
   IV. Sx: Horse No: 4 (New Jersey, U.S.), 14 year old Holsteiner mare.

Hx: Injured LF long pastern (PII) 12/15/11 jumping rail. Previously treated with laser, ECSW.
No improvement. Radiographs negative.

CS: 3/5 lame LF. Severe pain, swelling on cranio-lateral surface.

Dx: Periostitis of LF PII.

Tx: Local infiltration of 2 sq. in. area with 3×2.5 cc NOVEL (7/27/12).

Rx: 4 days post-injection (8/1/12): 25% improved swelling; 90% improved pain.
5 days post-injection (8/2/12): 100% sound. Resumed jumping.
30 days post-injection (8/30/12): 100% sound.
50 days post-injection (9/22130): remains sound!
   V. Sx: Horse No: 5 (Rome, Italy), 4 year old SB trotting mare.

Hx: Had fractured TMT as 2 year old. Chronically lame. Radiographs and I.A. cortisone one year ago.
No improvement. Lame for one year!

CS: 2.5/5.0 lame RF. Right carpus flexes sore. RE lateral splint palpates sore. Hocks sore.
Radiographs negative.

Dx: RF carpal DJD, RF lateral splint, tarsitis.

Tx: I.A. injection RRC/RIC (Carpus blocked 100% sound with carbocaine), RTT/RTMT and LTT/LTMT w with 2.5 cc NOVEL. RF lateral splint injected with 2.5 cc NOVEL.

Rx: 24 hours post-injection: no pain or swelling; 50% improvement of lameness.
96 hours post-injection: 1/5 lame on RE sore splint. (Splint injected with NOVEL and immediately non-painful).
8 days post-injection: Raced, finished 5^{th}. “Best race ever”. Only slightly on R line.
9 days post-injection: No fracture of splint on radiographs. Splint re-injected with NOVEL.
Immediately non-painful post-injection.
11 days post-injection: Raced again (3 days), finished 3^{rd}. Raced extremely well. Sound.

Race track closed in Rome month of August.

7 weeks post-injection: Raced, 2^{nd}.

9 weeks post-injection: Raced, 2^{nd}.

Per trainer, horse had been lame for more than one year and now has raced very well four times,

and has remained sound.

VI. Sx: Horse No: 6 (Rome, Italy), 4 year old SB trotting gelding.

Hx: Chronically lame. No improvement with numerous previous cortisone I.A. injections.

CS: 3/5 lame LF, with effusion and pain upon flexion of LFF.

DX: LFF synovitis.

Tx 1: I.A. injection LFF with 2.5 cc NOVEL. Traumatic tap!

Rx 1: 48 hours post-traumatic tap, slight swelling. No treatment.
96 hours post-injection, 80% improvement, able to train.

Tx 2: Trainer requests hock and stifle injections based on shoe wear.
LFF re-injected, LTT/LTMT, RTT/RTMT with 2.5 cc NOVEL/joint.
Stifles injected with 5.0 cc NOVEL/joint.

Rx 2: 48 hours post-injection: 90% improved per trainer.
Race track closed in Rome month of August.
2 months post-injection: raced, finished 4^{th}. Very good, per trainer.
   VII. Sx: Horse No: 7 (Rome, Italy), 4 year old SB trotting gelding.

Hx: Old RF mild tendon injury. RFF flexes sore. Sonogram O.K. Radiographs RFF negative.

CS: 2.5/5.0 swelling, pain, lameness RF.

Dx: Mild chronic tendonitis. Synovitis RFF.

Tx: I.A. injection RFF with 2.5 cc NOVEL.

RX: 72 hours post-injection: Raced, 5^{th}. Very good.

VIII. Sx: Horse No: 8 (Rome, Italy), 3 year old SB trotting filly.

Hx: Has never trained well. Unsound in front. Repeatedly breaks stride.

CS: Front fetlocks and knees flex sore.

Dx: Bilateral carpitis; synovitis front fetlocks.

Tx: I.A. knees, front fetlocks with NOVEL, 2.5 cc/joint (6 vials).

Rx: 48 hours post-injection, 50% improved per trainer.
96 hours post-injection, 80% improved per trainer.
Post-flexion, 100% non-painful.
Horse sold. Awaiting follow-up.
   IX. Sx: Horse No: 9 (Rome, Italy), 3 year old SB trotting gelding.

Hx: Lame one year. Everything injected with 1.A. cortisone in past. Nothing recently.

CS: On right shaft. RHF flexes 3+ sore.

Dx: Chronic lameness, generalized.

Tx 1: I.A. injection LHF with 2.5 cc NOVEL.

Rx 1: 4 days post-injection: trained straight in shafts, 50% improved. Needs shoe change

Tx 2: 48 hours after first injection: knees upper and lower, front fetlocks, front coffins lower hocks I.A. Novel 2.5 cc/joint.

Rx 2: 48 hours post-injections: 70% improvement per trainer.

X. Sx: Horse No: 10 (Rome, Italy), 4 year old SB trotting mare.

Hx: Qualified as a 2 year old in 2:04. Lame since, unraced, trains poorly. 1.A. cortisone injections 6
months ago with no improvement. Multiple lamenesses.

CS: Poor, lame trainer, makes multiple breaks. Right carpus flexes sore. Hocks sore.

Dx: R carpitis; bilateral tarsitis.

Tx: I.A. Novel RRC/RIC, LTMT?RTMT, 2.5 cc/joint.

Rx: 24 hours post-injection: Perfect, per trainer!
48 hours post-injection: Raced, 3^{rd}.
Track closed in Rome month of August.
2 months post-injected, trainer called, training perfectly!
   XI. Sx: Horse No: 11 (Rome, Italy), 4 year old SB trotting gelding.

Hx: Chronic lameness. Had laminitis all four feet in past. Chronic synovitis of ankles. Runs down
Behind.

CS: Effusion in all fetlocks. 3/5 lame in front.

Dx: Synovitis/O.A. all front fetlocks and front coffins. Synovitis hind fetlocks.

Tx 1: I.A. 2.5 cc NOVEL/fetlock.

Rx 1: 8 days post-injection: no effusion in fetlocks. Sound trotting in hand

Track closed month of August in Rome.

Tx 2: 7 weeks post first injections, LF/RF coffins and LFF/RFF injected 2.5 cc NOVEL/joint.

Rx 2: 48 hours post-injection: Minimal effusion; pain and lameness 50% improved.
6 days post-injection: Swelling, pain, lameness much improved. Training very well!
   XII. Sx: Horse No: 12 (Rome, Italy), 8 year old Quarter Horse (QH) barrel racing mare.

Hx: Chronic lameness, poor performance. Radiographs=DJD.

CS: 3/5 lame front fetlocks; sore hocks.

Dx: DJD hocks and front fetlocks.

Tx: I.A. 2.5 cc NOVEL injections LFF/RFF, LTT/LTMT, RTT/RTMT (6 vials).

Rx: 48 hours post-injection: 50% improvement in pain, swelling, lameness with flexion.
96 hours post-injection: 80% improvement in pain, swelling, lameness. Raced, did no good.
   XIII. Sx: Horse No: 13 (Rome, Italy), 12 year old QH barrel racing mare.

Hx: Chronic lameness, poor performance. OA of L stifle radiographically.

CS: 3/5 lame L hind.

Dx: OA of L stifle and hock.

Tx: I.A. 2.5 cc NOVEL LTT/LTMT; 5.0 cc NOVEL L stifle joint.

Rx: 48 hours post-injection: no effusion; 50% improvement in pain and lameness.
96 hours post-injection: 80% improvement in pain and lameness. Raced and won!
   XIV. Sx: Horse No: 14 (Rome, Italy), 12 year old QH aymkana mare.

Hx: Sore in front.

CS: 2.5/5.0 lame RF. Hoof test +; coffin joint effusion.

Dx: Pedal osteitis/synovitis.

Tx 1: I.A. 2.5 cc NOVEL front coffin joints.

Rx 1: 60% improvement of head nod RF.

Tx 2: 72 hours after initial injections: abaxial block RF foot with 2.5 cc NOVEL divided.

Rx 2: 48 hours post-foot block, no further improvement per trainer.
96 hours post-block, trainer states rode much better and didn't get tired as before!
   XV. Sx: Horse No: 15 (Papose, Italy), 3 year old SB trotting gelding.

Hx: Poor performance. Bad training.

CS: 2/5 lame L carpus, R hock, RH fetlock.

Dx: OA/DJD.

Tx: I.A. 2.5 cc NOVEL LRC/LIC, LTT/LTMT, LHF (5 vials).

Rx: 48 hours post-injection: no swelling or pain; 2/4 lame.
96 hours post-injection: 60% improvement in lameness.
10 days post-injection: 80% improvement. Trained well.
   XVI. Sx: Horse No: 16 (Massachusetts, U.S.), 10 year old SB pacing gelding.

Hx: Chronic, massive bowed tendon, one year old. Lame, unable to train. Has been on phenylbutazone.

CS: Very lame RE. Lays down in stall. 4/4 pain on palpation. 3/4 lame.

Dx: Tendonitis RE.

Tx: Intra-lesional injection of 20 cc NOVEL with 4 cc gentamycin. (Extensive surface area.)

Rx: 72 hours post-injection: swelling decreased 75% per trainer.
7 days post-injection: swelling decreased 90%. No pain or lameness. Back in training.
14 days post-injection: Raced and won (previously untrainable).
   XVII. Sx: Horse No: 17 (Massachusetts, U.S.), 10 year old SB pacing gelding.

Hx: Has had RF suspensory previously freeze fired. Lame RF.

CS: Distended volar pouch RF. 3/4 pain on flexion, 3/4 lame on lead shank, 1/4 lame on medial
suspensory branch.

Dx: RF suspensory desmitis and RFF synovitis.

Tx: I.A. Novel RFF.

Rx: 7 days post-injection: no effusion, no pain with flexion, 1/4 lameness. 2/4 pain lateral branch suspensory.
12 days post-injection: Raced and won in 1.56.4. (Raced all summer in 1:58).
14 days post-injection: no effusion, pain, or lameness.
17 days post-injection: Raced, won again!
   XVIII. Sx: Horse No: 18 (Massachusetts, U.S.), 6 year old SB pacing mare.

Hx: Old proximal PI fracture LF. Multiple I.A. injections with multiple medications. Multiple starts.
Owner “ready to Amish”.

CS: Chronic osselet LF. Very enlarged LFF. Effusion LF volar pouch, 4/4 pain on flexion, 3/4 lame
on lead shank.

Dx: Endstage DJD LFF with synovitis.

Tx: I.A. Novel LFF.

Rx: 7 days post-injection: decreased effusion, no pain on flexion. 1/4 lameness warms out of to 0/4.
8 days post-injection: Raced, finished 3^{rd} “Dramatic positive result”.
   XIX. Sx: Horse No: 19 (Massachusetts, U.S.), 3 year old SB pacing filly.

Hx: Poor performance.

CS: 1/4 lame in front. 2/4 positive for foot pain with hoof testers.

Dx: Sore front feet.

Tx: Perineural abaxial bocks front feet with NOVEL.

Rx: 7 days post injection, no swelling, pain, or lameness. Raced, won next start.
Horse raced next 6 weeks, finishing 1^{st} or 2^{nd} each start!
   XX. Sx: Horse No: 20 (Massachusetts, U.S.), 3 year old SB trotting gelding.

Hx: Making breaks, going off stride.

CS: 1/4 lame in front. 1/4 heel pain with hoof testers.

Dx: Sore front feet.

Tx: Peri-neural abaxial sesamoid injections bilaterally in front.

Rx: 7 days post-injection: No swelling, pain, or lameness.
Won next race.
   XXI. Sx: Horse No: 21 (Massachusetts, U.S.), 4 year old SB pacing gelding.

Hx: On left line racing.

CS: LF suspensory ligament 3/4 sore to palpation. 1/4 lame.

Dx: LF high suspensory desmitis.

Tx: Intra-lesional infiltration of suspensory with 10 cc NOVEL.

Rx: 7 days post-injection: no swelling, pain, or lameness. Racing.
14 days post-injection, racing.
21 days post-injection, racing.
28 days post-injection, racing.
   XXII. Sx: Horse No: 22 (Massachusetts, U.S.), 3 year old SB pacing gelding.

Hx: Hx: Hind end on right shaft.

CS: LH high suspensory. 3/4 painful on digital palpation, 2/4 swelling, 1/4 lame.

Dx: LH high suspensory desmitis.

Tx: Intra-lesional infiltration with 10 cc NOVEL.

Rx: 48 hours post-injection: 1/4 swelling, pain, lameness.
5 days post-injection: no swelling, pain, or lameness. Back in training.
   XXIII. Sx: Horse No: 23 (Massachusetts, U.S.), 7 year old SB pacing gelding.

Hx: Poor performance.

CS: Pain with foot palpation in front. 1/4 lame.

Dx: Front bilateral coffin joint synovitis

Tx: 2.5 cc NOVEL/joint.

Rx: 7 days post-injection: no swelling, pain, or lameness.
Raced, won next start.
   XXIV. Sx: Horse No: 24 (Massachusetts, U.S.), 10 year old SB pacing gelding.

Hx: 12 starts since 1/1/12. Monthly I.A. cortisone/Polyglycan injections. Lame LFF.

CS: LF volar pouch effusion, severe pain with flexion, 3/4 lame.Minimal ROM.

Dx: LFF arthritis, synovitis.

Tx: 7.5 cc NOVEL I.A. LFF.

Rx: 7 days post-injection: no swelling, pain on flexion, 2/4 lame.
21 days post-injection: no swelling, no pain on flexion. 1/4 lameness. Back in training.
   XXV. Sx: Horse No: 25 (Massachusetts, U.S.), 3 year old SB pacing gelding.

Hx: Racing lame.

CS: Distension of LRC/LIC joints. 3/4 lame LF. Painful on flexion.

Dx: LF carpitis.

Tx: I.A. NOVEL LRC/LIC joints.

Rx: 6 days post-injection: Raced, finished 3^{rd}.
7 days post-injection: decreased effusion, no pain with flexion, 2/4 lame.
14 days post-injection: swelling, pain, lameness all improved. Horse shipped elsewhere.
   XXVI. Sx: Horse No: 26 (Athens, Greece), 5 year old Thoroughbred (TB) mare.

Hx: Arthroscopic surgery of RIC joint 14 months ago. Severe DJD on radiographs. Sore and stiff
after every gallop. Previous I.A. injections with cortisone and HA.

CS: Lameness 1/5 after work. Swelling of RIC joint. Sore back.

Dx: DJD of RIC joint; RFL splint; back sore.

Tx: 2.5 cc NOVEL I.A. in RIC joint. 2.5 cc NOVEL locally infiltrated in splint. 10 cc NOVEL diluted into
30 cc normal saline for paralumbar infiltration of back. (Total of 14 cc NOVEL).

Rx: 24 hours post-injection: no swelling, pain, lameness.
48 hours post-injection: no swelling, pain, lameness.
60 hours post-injection: Raced, finished 3^{rd}, very close to winner with a 14 kg handicap.
Remarks: Trainer: She improved her time 1 second. She performs better at distance races
Rather than the 1400 meter race today. Never this pain free before.
Jockey: She was like a bird today.
Stable manager: 24 hours post-race, fantastic trotting, free like never before.
Vet: Amazing performance in this “big” race. Seems that the 10 ml diluted dose had a more general analgesic effect. Due to this the horse is pain free and can trot even after a hard race.
   XXVII. Sx: Horse No: 27 (Athens, Greece), 5 year old TB gelding.

Hx: Lameness post-gallop last 6 months. Mild DJD on radiographs.

CS: 1/5 lame RE.

Dx: DJD bilateral front fetlocks.

Tx: I.A. NOVEL 2.5 cc each front fetlock.

Rx: 24 hours post-injection: no swelling, pain, or lameness.
48 hours post-injection: no swelling, pain, or lameness.
7 days post-injection: Race, finished 4^{th} in race with major accident caused death of 2 horses.
8 days post-injection: immediately post-race and 24 hours later horse is better than ever.
   XXVIII. Sx Horse No: 28 (New Jersey, U.S.), 8 month old Paint gelding.

Hx: Acute tendon injury. Treated with phenylbutazoneand topical DMSO/dexamethasone.

CS: LF flexor tendonitis. 3/5 lame. Painful on flexion of ankle and knee. 1.5 times swelling.

Dx: Acute LF flexor tendonitis.

Tx: Injected tendon sheath with 5 cc NOVEL.

Rx: 7 days post-injection: much improvement in swelling, pain, lameness.
14 days post-injection: 80% improvement.
   XXIX. Sx: Horse No: 29 (New Jersey, U.S.), 17 year old TB gelding pleasure horse.

Hx: Chronically lame retired racehorse.

CS: Shifting hind end lameness. 3/5 lame, pain on stifle flexion, femero-tibial effusions.

Dx: Chronic femero-yibial synovitis.

Tx: 5 ml NOVEL/femero-tibial joint.

Rx: 5 days post-injection: swelling reduced 10%, 90% sound.
3 weeks post-injection: Sound. Back in use for flat work.
   XXX. Sx: Horse No: 30 (New Jersey, U.S.), 13 year old Welsh Pony gelding.

Hx: Lame after light jumping.

CS: Effusion and pain of front fetlocks. 3/5 lame.

Dx: Acute synovitis of front fetlocks.

Tx: I.A. injection 2.5 cc NOVEL/joint.

Rx: 5 days post-injection: 50% decrease in swelling. Pain and lameness much improved. Back to
full use with small jumping. Owner pleased.
   XXXI. Sx: Horse No: 31 (New Jersey, U.S.) 8 year old QH Paint gelding trail horse.

Hx: Acute RF lameness. Oral phenylbutazone administered.

CS: 4/5 lame RF. Suspensory twice normal size. Positive to palpation and flexion.

Dx: RF high suspensory desmitis.

Tx: Local infiltration with 5 cc NOVEL.

Rx: 6 days post-injection: 50% improvement in swelling, pain, and lameness.
3 weeks post-injection: 90% improved. Back to ring.
   XXXII. Sx: Horse No: 32 (New Jersey, U.S.), 17 year old Arabian gelding.

Hx: Acute lameness LF.

CS: Left carpal effusion. Reluctant to bear weight. 3/5 lame.

Dx: Acute carpal synovitis.

Tx 1: 5 cc NOVEL LRC/LIC joints.

Rx 1: 3 days post-injection: 50% improvement in effusion and pain. Sound. Back in training

Tx 2: I month later, reinjected with 5 cc NOVEL LRC/LIC joints.

Rx 2: 2 weeks post-injection: Sound.

XXXIII. Sx: Horse No: 33 (New Jersey, U.S.), 3 year old Arabian gelding.

Hx: Acutely 2/5 lame behind after dressage lesson. Phenylbutazonetwice daily for 2 weeks.

CS: Swelling and pain on flexion of hocks.

Dx: Acute distal tarsitis, spavin.

Tx: 1.A. injection of 2.5 cc NOVEL into LTMT/RTMT joints.

Rx: 48 hours post-injection: swelling reduced, no pain.
7 days post-injection: 90% sound.
2 weeks post-injection: returned to lesson work.
   XXXIV. Sx: Horse No: 34 (Rome, Italy), 2 year old SB trotting filly.

Hx: Acute lameness in training.

CS: 3/5 lame LH. 4/5 lame after L hock flexion test.

DX: Osteochondritis of L hock.

Tx: I.A. injection LTT/LTMT joints with 2.5 cc NOVEL/joint.

Rx: 48 hours post-injection: no swelling, 50% improvement in pain, 60% improvement of lameness.
5 days post-injection: 80% improvement of pain, 90% improvement in lameness. Raced and won!
2 weeks post-injection: no pain, no lameness. Trainer says very good, and has very good gait.
   XXXV. Sx: Horse No: 35 (Athens, Greece), 5 year old TB gelding.

Hx: Arthroscopic removal of apical fracture of LF lateral sesamoid. Previous I.A. injections of front fetlocks with cortisone and HA. Severe DJD front fetlocks on radiographs.

CS: Bilateral front end lameness. Synovitis front fetlocks.

Dx: DJD bilateral front fetlocks.

Tx: I.A. injection of 2.5 cc NOVEL each LFF/RFF.

Rx: 24 hours post-injection: 40% less swelling, 50% less pain, 30% improved lameness.
96 hours post-injection: 50% less swelling, 80% less pain, 80% improved lameness.
10 days post-injection: Raced, finished 2^{nd}. Post-race, “sounder than ever”.
19 days post-injection: Raced and won! Post-race, sound, very good.
   XXXVI. Sx: Horse No: 36 (Athens, Greece), 4 year old TB gelding.

Hx: RF distal cannon bone fracture surgically repaired with 2 screws. Previous I.A. injection with cortisone and HA.

CS: mild RFF synovitis.

Dx: Synovitis of RF fetlock.

Tx: I.A. 2,5 cc NOVEL in RFF.

Rx: 48 hours post-injection: 80% improvement in swelling.
10 days post-injection: Raced, finished 2^{nd} in dead heat. No pain, swelling or lameness.

Remarks(vet): Very difficult race against best horses at the track!

XXXVII. Sx: Horse No: 37 (Athens, Greece), 3 year old TB colt.

Hx: Surgical repair of LF displaced distal cannon bone fracture with 3 screws. DJD of LFF radiographically.

CS: 1/5 lame LF. Capsulitis of LFF. Pain on flexion. Effusion.

Dx: DJD of LF fetlock.

Tx: 2.5 cc NOVEL I.A. LFF.

Rx: 3 days post-injection: 40% less swelling, 80% less pain, 60% improvement in lameness. Sound in training. There was no need for pain medication after injection through the capsulitis, which has been the usual.

XXXVIII. Sx: Horse No: 38 (Athens, Greece), 3 year old TB filly.

Hx: Lameness of RE, becoming worse after work.

CS: 1/5 lame RF. Bilateral front fetlock synovitis.

Dx: DJD bilateral front fetlocks.

Tx: 2.5 cc NOVEL 1.A. each LFF/RFF.

Rx: 3 days post-injection: 80% improvement in swelling, pain, and lameness.
7 days post-injection: 80% improved swelling, no pain, no lameness.
Remarks (trainer): Very happy. Horse not lame after work. Much improvement over injections with cortisone and HA. Ready to race!
   XXXIX. Sx: Horse No: 39 (Athens, Greece), 4 year old TB gelding.

Hx: Surgical repair of displaced RF distal cannon bone fracture with 3 screws. Previous I.A. cortisone and HA injections. Always 4/5 lame after inject. Severe DJD radiographically.

CS: 2/5 lameness RF. RFF synovitis, capsulitis.

Dx: DID RF fetlock.

Tx: 2.5 cc NOVEL 1.A. RFF.

Rx: 3 days post-injection: swelling the same, pain and lameness 60% improved.
Horse was non-painful post-injection and did not need usual pain medication.
   XL. Sx: Horse No: 40 (Athens, Greece), 2 year old TB gelding.

Hx: Acutely lame RF.

CS: Acute swelling RF tendon. Sonogram: core lesion in 2A-2B of RF SDFT.

Dx: Acute superficial digital flexor tendonitis (SDFT).

Tx: 6.0 cc NOVEL intra-lesionally via ultrasound guidance.

Rx: 24 hours post-injection: 50% less swelling, pain, and lameness.
72 hours post-injection: 80% improvement in Swelling, pain, and lameness.
12 days post-injection: trainer applied a “blister” to the leg. There was no reaction, i.e. no significant swelling or inflammation!
35 days post-injection: 90% less swelling, no pain, no lameness.
Ultrasound exam: “Remarkable healing of lesion”.
   XLI. Sx: Horse No: 41 (New Jersey, U.S.), 24 year old Arabian mare pleasure horse.

Hx: Hock issues. Has become progressively lamer in hocks with age.

CS: Stiff behind. 4/5 hock lameness, left worse than right.

Dx: Chronic osteoarthritis bilateral hocks.

Tx: I.A. 2.5 cc NOVEL each LTMT, RTMT joints.

Rx: 48 hours post-injection: 20% improvement (3/5).
1 week post-injection: 20% improvement (3/5).
2 weeks post-injection: Sound after first week. Moves well. Negative to flexion.
3 weeks post-injection: used for rough trail ride and is 100% sound!
   XLII. Sx: Horse No: 42 (New Jersey, U.S.), 20 year old Appaloosa gelding pleasure horse.

Hx: Used heavily as school horse for 10 years. Progressive arthritic hock problems. Right hip issue.

CS: 3/5 hock lameness, right worse than left. Sore right hip.

Dx: Chronic osteoarthritis bilateral hocks.

Tx: I.A. 2.5 cc NOVEL each LTMT, RTMT joints.

Rx: 48 hours post-injection: 3/5 lame. No noticeable improvement by caretaker.
1 week post-injection: 2/5 lame. Favors right hind. Most likely untreated right hip.
2 weeks post-injection: Back in continuous work. Much improvement in gait and to flexion. No further treatment necessary.
   XLIII. Sx: Horse No: 43 (New Jersey, U.S.), 14 year old TB gelding pleasure horse.

Hx: Injured when another horse fell on his back. Only able to be ridden 10 lessons.

CS: Decreased performance. 4/5 painful to digital palpation of epaxial musculature.

Dx: Thoracolumbar inflammation (back sore).

Tx: Intramuscular (I.M.) injection of epaxial muscles with 10 cc NOVEL+10 cc saline divided into vfive 4 cc injections per site for each side.

Rx: 48 hours post-injection: No noticeable improvement. 4/5 painful to palpation.
1 week post-injection: 3/5 painful to palpation.
2 weeks post-injection: 2/5 painful to palpation. Improvement slow, but at least 50% better over time.
   XLIV. Sx: Horse No: 44 (New Jersey, U.S.), 14 year old TB mare pleasure horse.

Hx: Ex-race horse with back issues. Recovering from Lyme's disease, out of work for 4 months.

CS: Decreased performance. Muscle loss of topline. 3/5 painful to palpation of epaxials.

Dx: Thoracolumbar inflammation (sore back).

Tx: I.M. injection of epaxial muscles with 10 cc NOVEL+10 cc saline divided into five 4 cc
injections per site for each side.

Rx: 48 hours post-injection: 2/5 painful to palpation.
1 week post-injection: 2/5 painful to palpation.
2 weeks post-injection: 90% improvement. Occurred gradually. “Wonderful flowing gait”!
   XLV. Sx: Horse No: 45 (New Jersey, U.S.), 16 year old Arabian gelding pleasure horse.

Hx: Ex-race horse with back issues.

CS: Decreased performance. 3/5 painful to digital palpation of epaxial musculature.

Dx: Thoracolumbar inflammation (back sore).

Tx: I.M. injection of epaxial muscles with 10 cc NOVEL+10 cc saline divided into five 4 cc injections
per site for each side.

Rx: 48 hours post-injection: 2/5 pain left side; 3/5 pain right side.
1 week post-injection: 2/5 painful to palpation.
2 weeks post-injection: 1.5/5 painful. Approximately 50% improvement. “Horse has many other chronic problems. NOVEL made a difference”.

| | Number of horses |
|---|---|
| AICAR injection site | injected |
| Intra-articular | |
| Coffin joints (distal interphalangial) | 13 |
| Fetlock (ankle) joints (metacarpal-phalangial/ | 31 |
| metatarsal-phalangial) | |
| Hock joints (tibial-tarsal/tarso-metatarsal) | 34 |
| Stifle joints (femeral-tibial) | 5 |
| Carpal (knee) joints (radial-carpal/intercarpal | 21 |
| or carpal-metacarpal) | |
| Foot blocks (abaxial sesamoids) | 6 |
| Suspensory ligaments | 5 |
| Osteitis | 1 |
| Splints (exostoses) | 2 |
| Tendons (2 acute/1 chronic) | 3 |
| Backs | 4 |

Table one above summarizes the experimental localized usage of NOVEL (AICAR) set forth in the Examples above.

What is claimed is:

1. A composition adapted for the localized treatment and/or prevention of a disease condition or traumatic injury in a subject comprised of a therapeutically effective amount of a suitable agonist of AMP-activated protein kinase (AMPK).

2. The composition of claim 1, wherein the disease or injury is musculoskeletal.

3. The composition of claim 1, wherein the subject is a mammal.

4. The composition of claim 3, wherein the mammal is a selected from the group consisting of a human, an equine, a bovine, a caprine, an ovine, a porcine, a cervidae, a canine, a feline, a non-human primate or a camelid.

5. The composition of claim 2, wherein the musculoskeletal disease or injury is selected from the group consisting of osteoarthritis, exostoses, osteitis, periostitis, synovitis, bursitis, capsulitis, tendonitis, desmitis, a ligament strain, a ligament tear, a tendon strain, a tendon tear, a muscle strain, a pulled muscle, a muscle tear and the like.

6. The composition of claim 1, wherein the localized delivery is selected from the group consisting of topical treatment directly to or adjacent the site of disease or injury; direct injection at the site of the disease or injury; or local infiltration of the composition adjacent the site of disease or injury.

7. The composition of claim 1, wherein the composition is a fluid.

8. The composition of claim 7, wherein the fluid is non-Newtonian.

9. The composition of claim 1, further comprising a therapeutically effective amount of an agent selected from the group consisting of: hyaluronic acids; sulfated polysaccharides (e.g. chondroitin sulfate, polysulfated glycosaminoglycans); and pentosan polysulfate; glycosaminoglycan peptide complexes); N-acetyl-D-glucosamine; N-acetyl-D-glactosamine; glucosamine sulfate; glucosamine HCl; corticosteroids (e.g., methylprednisolone acetate, betamethasone, triamcinalone acetonide, isoflupredone acetate and dexamethasone); non-steroidal anti-inflammatory agents (e.g., bufexamac, ketoprofen, naproxen, ibuprofen, meloxicam, flunixin meglumine, carprofen, phenylbutazone, ketoprofen, firocoxib and deracoxib); local anesthetics (e.g., mepivacaine and lidocaine); superoxide dismutase; dimethyl sulfoxide; autologous conditioned serum; autologous conditioned plasma; platelet rich plasma; interlukin-1 receptor antagonist protein (e.g., IRAP I and IRAP II); stem cells (e.g., mesenchymal stern cells, bone marrow derived stem cells, umbilical cord-derived stem cells, and cultured stem cells); chondrocytes; insulin like growth factor-1 (IGF-1); lubricin/proteoglycan 4/PRG4; gene therapy products; nanoparticles; pitcher plant extract (e.g., SARAPIN and P-BLOC); and combinations thereof.

10. The composition of claim 1, wherein the suitable AMPK agonist is AICAR.

11. The composition of claim 10, wherein the therapeutically effective amount is from between about 0.01 mg/ml to about 400 mg/ml.

12. The composition of claim 10 wherein the therapeutically effective amount is from between about 1 mg/ml and about 20 mg/ml.

13. The composition of claim 1, wherein the suitable AMPK agonist is selected from the group consisting of AICAR, metformin, phenformin, A-769662, resveratrol, thiazolidindiones (including rosiglitazone, pioglitazone and troglitazone), D942, S27847, nootkatone, berberine, dhberberine, polyphenols (including S 17834, piceatannol, CA-4, EGCG, TF1,TF2,TF3), WS070117, leptin, adiponectin, DRL-16536, BG800, MT-39 series of structures, salicylic acid (its salts and prodrugs), triterpenoids (including cucurbitane triterpenoids and ginsenoside Rg3) and combinations thereof.

14. The composition of claim 10, wherein the composition is a fluid.

15. The composition of claim 10, wherein the fluid is non-Newtonian.

16. The composition of claim 1, further comprising a suitable hyaluronic acid.

17. The composition of claim 16, wherein the suitable hyaluronic acid has a molecular weight of from between about 100 thousand Daltons to about 6.5 million Daltons.

18. The composition of claim 16, wherein the suitable hyaluronic acid has a molecular weight of from between about 300 thousand Daltons to about 3.5 million Daltons.

19. The composition of claim 16, wherein the suitable hyaluronic acid has a molecular weight of from between about 500 thousand Daltons to about 1.5 million Daltons.

20. The composition of claim 16, wherein the concentration of the hyaluronic acid is from between about 5 mg/ml to about 10 mg/ml.

21. The composition of claim 16, wherein the concentration of the hyaluronic acid is greater than about 10 ma/ml.

22. The composition of claim 16, wherein the concentration of the hyaluronic acid is less than about than about 5 mg/ml.

23. A method of treatment of osteoarthritis in a mammal comprised of administering a therapeutically effective amount of the composition of claim 1 to the mammal.

24. The method of claim 23, further comprising the intra-articular administration of a therapeutically effective amount of the composition of claim 1 to the mammal.

25. A method of treatment of damage to tissues of the musculoskeletal system in a mammal comprised of administering a therapeutically effective amount of the composition of claim 1 to the mammal.

26. The method of claim 25, further comprising the localized administration of a therapeutically effective amount of the composition of claim 1 to the damaged tissues of the musculoskeletal system in the mammal.

27. A composition adapted for direct intravesical instillation in to the bladder of a mammal comprised of a therapeutically effective amount of a suitable AMPK agonist.

28. A method of treatment of cystitis in a mammal comprised of administering a therapeutically effective amount of the composition of claim 27 to the mammal.

29. A composition adapted for use as a medical device useful as a fluid replacement for ophthalmic surgical procedures comprised of a therapeutically effective amount of a suitable AMPK agonist.

30. The composition of claim 29, further comprising a therapeutically effective amount of a suitable HA.

31. A composition adapted for the intra-articular treatment and/or prevention of osteoarthritis in a mammal comprised of a therapeutically effective amount of AICAR and a suitable hyaluronic acid.

32. The composition of claim 31, wherein the composition is a non-Newtonian fluid.

33. A composition adapted for localized (e.g., intra-lesional) treatment of damage or disease to connective tissue in a mammal comprised of a therapeutically effective amount of AICAR and a suitable hyaluronic acid.

34. A composition adapted for local administration for providing analgesia at a pre-selected site in a subject comprised of a therapeutically effective amount of a suitable agonist of AMP-activated protein kinase (AMPK).

35. The composition of claim 34 wherein the suitable AMPK agonist is AICAR.

36. The composition of claim 34, wherein the therapeutically effective amount is from between about 0.01 mg/ml and about 400 mg/ml.

37. A method for providing local analgesia at a preselected site in a mammal comprising administering a therapeutically effective amount of the composition of claim 34 into or adjacent the preselected site in the mammal.

38. A method of treatment of laminitis in a horse comprised of administering by localized delivery a therapeutically effective amount of the composition of claim 1 to the affected tissue in the horse.

39. The method of claim 38, wherein the composition of claim 1 is administered to via regional intravenous perfusion (retrograde perfusion).

40. The composition of claim 38, wherein the suitable AMPK agonist is AICAR.

41. A method of treatment of EPM in a horse comprised of administering by localized delivery a therapeutically effective amount of the composition of claim 1 to the horse.

42. The method of claim 41, wherein the composition of claim 1 is administered to into the CSF via epidural injection.

43. The method of claim 41, wherein the suitable AMPK agonist is AICAR.

44. A composition adapted for local administration that is capable of acting as an analgesic and an anti-inflammatory at a pre-selected site in a mammal comprising a therapeutically effective amount of a suitable AMPK agonist.

45. The composition of claim 44 wherein the suitable AMPK agonist is AICAR.

46. The composition of claim 45, wherein the therapeutically effective amount is from between about 0.01 mg/ml and about 400 mg/ml.

47. A method for simultaneously providing local analgesia and treatment and/or prevention of inflammation at a preselected site in a mammal comprising administering a therapeutically effective amount of the composition of claim 44 into or adjacent the preselected site in the mammal.

48. The method of claim 47, wherein the preselected site is a surgical incision.

49. The method of claim 47, wherein the preselected site is the margin of an open wound.

50. A medical device adapted for intra-articular administration for use as a surgical lavage during or after surgical procedures in a mammal comprising a therapeutically effective amount of a suitable AMPK agonist.

51. The medical device of claim 50, further comprising a therapeutically effective amount of a suitable HA.

52. The medical device of claim 51, wherein the suitable AMPK agonist is AICAR.

53. A medical device adapted for topical use for treatment of a wound in a mammal comprising a therapeutically effective amount of a suitable AMPK agonist.

54. The medical device of claim 53, further comprising a therapeutically effective amount of a suitable HA.

55. The medical device of claim 53, wherein the suitable AMPK agonist is AICAR.
